(19) United States
(12) Patent Application Publication
(10) Pub. No.: US 2009/0311785 A1
(43) Pub. Date: Dec. 17, 2009

(54) Method for organizing and controlling cell growth and tissue regeneration

(76) Inventors: Luis Nunez, Elmhurst, IL (US); Gustavo Larsen, Lincoln, NE (US); Eduardo Mansilla, La Plata (AR); Hugo Alberto Drago, Buenos Aires (AR)

Correspondence Address: LOUIS VENTRE, JR, 2483 OAKTON HILLS DRIVE, OAKTON, VA, 22124-1530, US

(21) Appl. No.: 12/544,307

(22) Filed: Aug. 20, 2009

Related U.S. Application Data
(63) Continuation-in-part of application No. 11/743,560, filed on May 2, 2007.
(60) Provisional application No. 60/746,311, filed on May 3, 2006.
(60) Provisional application No. 60/886,225, filed on Jan. 23, 2007.

(51) Int. Cl. C12N 5/08

(52) U.S. Cl. 435/396

(57) ABSTRACT

A method for organizing and controlling cell growth on a cell-free structure comprises steps of decellularizing a biological material to produce an acellular matrix; adding cells sought to be propagated to the acellular matrix; coating the acellular matrix with a fiber comprising biological and nonbiological material; and storing the covered acellular matrix for a time sufficient to form organized cell growth in the three-dimensional structural shape. The method may include the step of covering the three-dimensional structural shape by a structural layer to enable generation of a replacement external organ. This structural layer comprises a polymer film and fiber with a biological material and a nonbiological material. The biological material includes a cell growth factor; and the nonbiological material includes magnetite. This structural layer also comprises a therapeutic at a concentration between 0.001 and 5 weight percent. This structural layer may also comprise an acellular matrix of an organ.

### CROSS-REFERENCE TO RELATED APPLICATIONS

This application is a continuation-in-part of U.S. application Ser. No. 11/743,560, filed May 2, 2007, which claims the benefit of U.S. Provisional Application No. 60/746,311, filed May 3, 2006, and U.S. Provisional Application Ser. No. 60/886,225, filed Jan. 23, 2007, the disclosures of which are expressly incorporated herein by reference in their entirety.

### TECHNICAL FIELD

In the field of artificial substitutes or parts for a human body particularly manufactured or adapted from natural living tissue to replace or assist a missing or defective natural body member or part thereof for functional or cosmetic reasons and to simulate multi-dimensional structures for diagnostic and/or tissue or organ simulations and studies.

### BACKGROUND ART

This invention may be utilized to provide significant advancements applicable to four bioengineering applications: (1) acute & chronic non healing wounds, (2) organogenesis and regenerative medicine, (3) three-dimensional (3-D) tissue and organ diagnostic models and (4) tumor engineering and treatment.

The present invention is new and innovative in that it provides needed stimulation, organization and control of the cell-growth process using a cell-free structure composited with fiber and cells and in some embodiments polymer film. The combination enables stem cells, for example, to reach and fix to a structure derived from a bloodstream, bone marrow, umbilical cord, or other source such as from tissue engineering laboratories, and, as a consequence, grow faster and in a particular direction after populating the structure.

Bioengineered materials have proved effective for a variety of tissue-engineering applications, facilitating studies in the repair of cartilage, bone, skin healing and others. Delivered by patch, injection, or implantation, or other methods, such materials can be used in multiple formats, including electrohydrodynamic (EHD), hydrogels, lyophilized hydrogel sponges, dried hydrogel films. However, existing materials suffer from numerous deficiencies solved by the present invention.

The use of acellular matrix grafts of collagen and elastin fiber is disclosed in U.S. Pat. No. 7,306,627. The procedure taught is one using an acellular matrix graft isolated from natural sources and consist essentially of a collagen and elastin matrix which is devoid of cellular components. The '627 patent teaches that the grafts are useful scaffolds which promote the regeneration of muscle tissue and aid in restoring muscle function. Due to their acellular nature, the grafts lack antigenicity. As a result, the acellular matrix grafts can be isolated from autographic, allographic or xenographic tissues. However the elastin fibers and collagen fibers are not those of the present invention that are added as a separate entity and crafted to contain nonbiologic material and biologic material. Rather, the '627 patent teaches that the collagen and elastin fiber is an inherent part of the acellular matrix.

The fibers used in the present invention derive from those described in the parent application, U.S. application Ser. No. 11/743,560, Publication No. US20070296099. They are specially formed to contain both biological material and nonbiological material. The polymer film used in an embodiment of the present invention is also a unique structural layer used in the invention.

### Acute & Chronic Non Healing Wounds

It is well known that abnormal physiological environments result in failure to promote wound healing, such as from burns, or venous, diabetic, and pressure ulcer wounds. Abnormal physiological environments also result in healing leading to deformities. When impairments in wound healing arise, it is often followed by an increased susceptibility to infection. Also, these failures result in a patient experiencing further ailments from complications in damage or failure of other organs. The present invention is useful to promote healing of a wound infection, lower impairments in healing, and lower the risk of subsequent complications.

An example of potential use of the invention is associated with people with diabetes. In 2006 in the US, there were over 800,000 diabetic patients with diabetic ulcers, and 1.5 million patients with pressure ulcers. Diabetes is the most common disease process associated with lower limb amputation, accounting for nearly half of all non-traumatic amputations in North America and Europe. In the United States, 162,500 patients with diabetes are hospitalized for foot ulcers annually.

Of the 50,000-60,000 diabetes-related lower-extremity amputations performed every year, 84% are preceded by a foot ulcer. Equally important, once people with diabetes develop an open wound, they are further hampered both by impairments in wound healing and by an increased susceptibility for wound infection. Two to three percent of all patients with diabetes develop foot ulcers every year, and approximately 15% develop foot ulcers during their lifetime.

Not only is the risk for amputation increased in patients with diabetes, but the mortality rate is significantly higher among this population as well. Complications involving incidence of ischemic heart disease, arterial disease, and peripheral vascular disease is twice as high among this population. These complications have been shown to be the cause of the majority of mortalities associated with amputation. Patients with diabetes admitted to a nursing home with a pressure ulcer had an 88% greater death rate by one year than non-diabetics with similar foot wound infections. An invention, such as the present invention, that can promote healing and thus lower the risk of complications for diabetics would offer a significant treatment remedy not presently available.

The present invention is applicable to pressure ulcers or “bed sores,” which are a serious medical condition that results in the destruction of skin tissue. Approximately 10% of all hospital patients are afflicted by pressure sores, which may ultimately lead to pressure ulcers.

Studies have shown that distinctive growth factors and cytokines are released by the keretinocytes and fibroblasts cells in tissue-engineered skin at the proper concentration and ratios to effectively induce repair while providing the proper extra-cellular matrix (ECM) components.

Chronic non-healing wounds can become a portal for bacterial entry into the body and subsequent infection. In the case of diabetic ulcers, chronic infections are usually polymicrobial, involving multiple aerobic and anaerobic pathogens. Physiologically, wound healing can be divided into three stages: inflammation, proliferation, and remodeling.

Wound repair is characterized by a series of complex cellular and molecular events. Numerous growth factors are involved in these processes and act by stimulating chemotaxis, cellular proliferation, extracellular matrix formation, and angiogenesis, with contraction and reestablishment of cellular integrity. The present invention enables combining a specific combination of growth promoting substances in a single matrix, substantially simplifying the treatment process.

Chronic wounds tend to involve lower amounts of growth factors when compared to acute wounds. Cytokines and metallomatrix proteins are believed to increase destruction and/or inhibition of growth factors and induced repeated trauma and infection. Present chronic wound treatments are insufficient in preventing scar formation and promoting healing. The present invention enables a treatment that will minimize scar formation while promoting healing.

### Organogenesis and Regenerative Medicine

Regenerative medicine is that which seeks to regenerate damaged tissue and organs by stimulating the damaged tissue and organs to repair themselves by the use of cells.

The field of organogenesis is faced with several challenges: the choice of the most appropriate cells for each application, the methods for their proliferation and differentiation, and the necessary knowledge related to the best matrices (e.g., tissue scaffolds) to be used. The numerous biological signals and molecular pathways that shape the particular architecture of specific organs, like the kidney or the liver, are not yet completely understood.

Organ transplant waiting lists are growing all over the world and it is nearly impossible to modify this tendency, at least not in the foreseeable future. Also, chronic rejection is not a rare complication after organ transplantation.

Regenerative medicine is a relatively new discipline. In one of its forms, it uses tissue engineering techniques and stem cells for production of simple tissues. Little is known about the role of insoluble factors of the extracellular matrix that mediate the phenotypic differentiation of stem cells. The biophysical and electrical nature of the cellular microenvironment, its geometrical configurations, and its biochemical properties can definitively influence the development of three-dimensional (3D) multicellular morphogenesis.

An important benefit of the present invention is that it enables stem cells, to be cultured in a 3D microenvironment. Examples of important stem cell types are Mesenchymal stem cells (MSCs), adult stem cells, and embryonic stem cells. The 3D microenvironment is an acellular matrix derived from an organ or a tissue. This acellular matrix enables organ or tissue development into similar phenotypes as those of the cells present in the original organ or tissue from which the acellular matrix had been obtained. Organ development into a similar phenotype is a unique benefit that enables regeneration of that specific organ. In this way, the invention has an important use as a means to provide full-complexity and functional organs employing an Intelligent Acellular Xenogeneic Isomorphic Matrix (IAXIM), MSCs and other stem cells.

### Three-Dimensional (3-D) Diagnostic Models and Tumor Engineering

The present invention provides a means to deliver a therapeutic or an imaging agent and its vehicle, vector or carrier in order to maximize treatment and imaging of malignant cancers in patients while minimizing the adverse effects of treatment. Selective delivery of therapeutic agents to a desired part of the body is also a nontrivial issue. Current treatments may lead to insufficient tumor distribution or therapeutic agents and often cause adverse effects on patients. Systemic injections of therapeutic agents carry consequences associated with their nonspecific dispersion in the body and have a limited therapeutic agent distribution throughout the targeted malignancy. Using the method of the invention, one can design an effective therapeutic or imaging agent delivery vehicle.

Similarly, acellular tumor matrices with fibers as described for use in the invention can be used for therapeutic and/or diagnostic purposes.

### SUMMARY OF INVENTION

A method for organizing and controlling cell growth on a cell-free structure comprises a step of decellularizing a biological material to produce an acellular matrix. The acellular matrix is a three-dimensional structural shape desired for cell growth. It comprises a step of adding cells sought to be propagated to the acellular matrix. It comprises a step of coating the acellular matrix with a fiber. The fiber comprises biological and nonbiological material. The method comprises a step of storing the covered acellular matrix for a time sufficient to form organized cell growth in the three-dimensional structural shape. The method may include the step of covering the three-dimensional structural shape by a structural layer to enable generation of a replacement external organ. This structural layer comprises a polymer film and fiber with a biological material and a nonbiological material. The biological material includes a cell growth factor; and the nonbiological material includes magnetite. This structural layer also comprises a therapeutic at a concentration between 0.001 and 5 weight percent. This structural layer may also comprise an acellular matrix of an organ.

When used for regenerating skin organ, the method includes a step of covering the coated acellular matrix with fibers on a polymer film wherein the covering is configured to limit growth of a skin organ (170). In this embodiment the polymer film and the fiber covering the acellular matrix are configured to limit growth of a skin organ.

### TECHNICAL PROBLEM

In all cases in the prior art, the development of effective treatment options using bioengineered materials requires improved 3D cell cultures to better simulate living systems. Current, culture environments are limited by their inability to mimic the micro environment of cells and tissues.

Technology for regenerative medicine concentrates on seeding a biodegradable scaffold with cells of a particular tissue type. More recent efforts have focused on tissue growth and regeneration to occur in vivo rather than ex vivo. However, all current regeneration therapies employ a complex process of a combination of scaffold, cells, and regulatory molecules.

### SOLUTION TO PROBLEM

The present invention solves this problem by creating a unique environment using an acellular matrix coated with fiber comprising a combination of nonbiological material and biological material in a culture that promotes the attachment, growth and differentiation of cells.

The acellular matrix of the present invention is a coated carrier providing the structure and environment for cell growth, which stimulates tissue and organ regeneration significantly more effectively than the prior art. It is referred to herein as an acellular matrix coated carrier.

### ADVANTAGEOUS EFFECTS OF INVENTION

The present invention provides a single matrix that is able to anchor the proper cells, delivery the regulatory molecules, deliver therapeutic agents, and control the moisture and temperature of the healing tissues under one umbrella to maximize healing and/or organ growth.

An acellular matrix coated carrier in accordance with the present invention will mimic the in vivo mechanical environment and consequently will condition and stimulate the development of cells and tissues. Use of an acellular matrix coated carrier provides new methodology that will enable expanding academic and industrial research and development.

By enabling biological growth systems to mimic the in vivo mechanical environment, the present invention enables biologists to condition and engineer developing tissues, reveal basic regulatory pathways and mechanisms of cell function, direct stem cell differentiation, provide an in vitro system for drug development. Such in vitro system may include changing the gene profile of cells, such as those found in normal or neoplastic tissues.

An embodiment of the invention enables stem cells, such as Mesenchymal stem cells, adult stem cells or embryonic stem cells, to concentrate and influence the 3D multicellular morphogenesis while overcoming the limitations and lack of selective attachment with the current technology.

### BRIEF DESCRIPTION OF DRAWINGS

FIG. 1 is a diagram of the steps in preferred embodiments of the invention.

FIG. 2 is a cross-section of an exemplary fiber used in a preferred embodiment of the invention.

### DESCRIPTION OF EMBODIMENTS

In the following description, reference is made to the accompanying drawing, which forms a part hereof and which illustrates several embodiments of the present invention. The drawings and the preferred embodiments of the invention are presented with the understanding that the present invention is susceptible of embodiments in many different forms and, therefore, other embodiments may be utilized and structural, and operational changes may be made, without departing from the scope of the present invention. The steps shown and described herein may be performed in any order that results in the desired cell growth. In the drawing, optional steps are shown with dotted arrows.

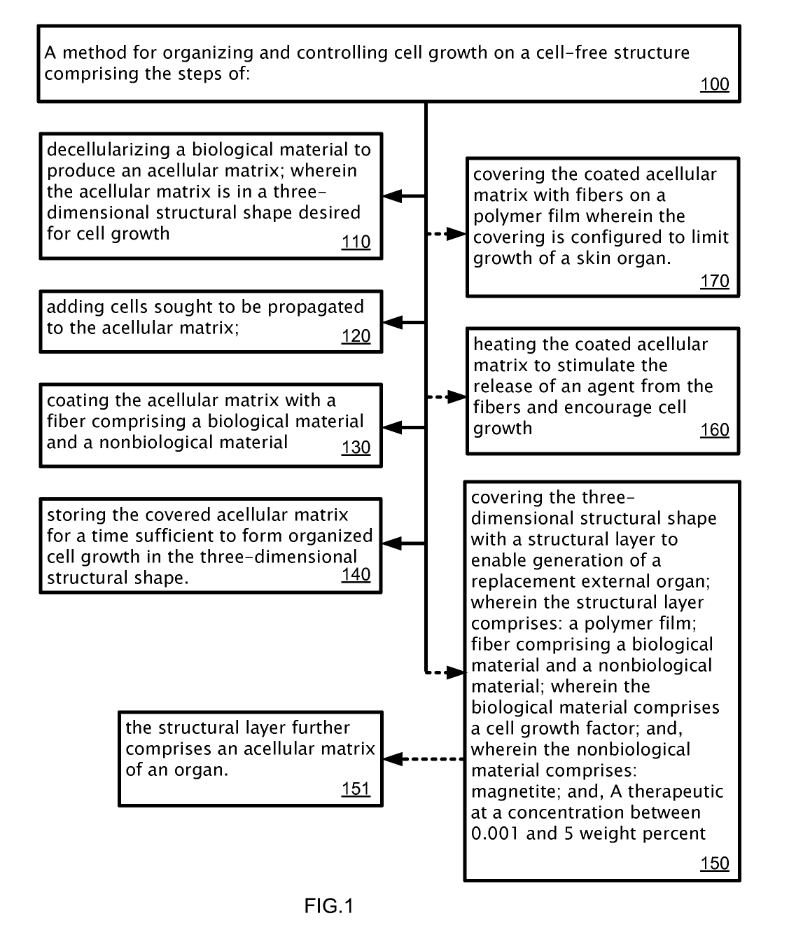

A preferred embodiment of the present invention is a method for organizing and controlling cell growth on a cell-free structure (100), shown in FIG. 1. For example, it may be used for tissue growth and organ regeneration.

A preferred method of the invention employs a bioengineered material constructed from an acellular matrix coated in three dimensions with a cell growth culture. Therefore, a step in the method comprises decellularizing a biological material to produce an acellular matrix; wherein the acellular matrix is in a three-dimensional structural shape desired for cell growth (110).

A biological material is any tissue or organ originating from an animal. The biological material is typically obtained from one species for use in another. It may also be obtained from an individual of a species for use in another individual of the same species.

The step of decellularizing a biological material is performed by chemically or bio-chemically destroying all the cells in the biological material, that is, all the cells in the tissue or organ. The acellular matrix formed in the decellularizing step forms a three-dimensional structural shape desired for cell growth.

Examples of biological material are a pig's skin (porcine), liver, heart, etc. For pig's skin, an example of decellularizing that biological material, is taking a portion of the pig's skin and chemically or bio-chemically destroying all the cells in that portion of the skin. What remains is the framework of the organ in which pig cells populated and generated what was the pig's skin. This framework is organic in nature and was originally generated as part of the organ producing process over a relatively long period of time. It tends to be inert as well as non immunogenic, and, in the present invention, is a structure or home for new cells from, for example, a human to populate. Since the framework is organic in nature, it may be used on a human host without inducing an immuno-response.

A preferred method of the invention next comprises a step of adding cells sought to be propagated to the acellular matrix (120). Examples of cells to be propagated are stem cells (such as Mesenchymal stem cells, multipotent adult stem cells, and embryonic stem cells), progenitor cells, stromal cells, and cells from a person's open wound that must be healed, an organ that needs to be regenerated, or a tumor that must be engineered. Organ cells, for example, include keretinocytes and fibroblasts cells in tissue-engineered skin.

Both stem and progenitor cells are thought to differentiate into new tissue, enable recruitment of additional stem or progenitor cells, and/or secrete a combination of molecules enabling endogenous repair events. Stem and progenitor cells are present in the blood and most tissues, and are mobilized to the injury site by acute injury signals, such as acute inflammatory cytokines. Tumor necrosis factor (TNF), Platelet Growth Factor, Epidermal Growth Factor, Granulocyte-Macrophage Colony Stimulating Factor are examples of such inflammatory cytokines. Prior studies have demonstrated that only rare numbers of these stem or progenitor cells can be found in the healing tissue, often in insufficient number to contribute to appropriate tissue repair and healing.

The present invention is adaptable to build up complex organs using stem cells in vivo as well as ex vivo. Mesenchymal stem cells (MSCs) display great flexibility to differentiate into many cell types, which essentially depends on the medium in which they are embedded. They can differentiate to mesoderm lineages and also to neuroectoderm and endoderm type cells. They also seem to be nonimmunogenic. This last attribute makes them especially suitable for allogeneic strategies. They appear to be permanently contributing to organ regeneration, especially during disease or injury. If MSCs are placed in a specific, defined “embryonic niche” to allow exposure to the repertoire of organogenic signals, they could in principle generate full organs. The present invention supplies this “embryonic niche.”

A preferred method of the invention next comprises a step of coating the acellular matrix with a fiber comprising a biological material and a nonbiological material (130). The biological and a nonbiological material are preferably added when the fiber is made by electrohydrodynamics in a core shell manner as described in U.S. Patent Publication US20070296099 (U.S. patent application Ser. No. 11/743,560), which is incorporated by reference herein. However, distinct core and shell regions for the fiber are not required.

The fiber coating adds materials to the acellular matrix to anchor stem cells and start growth and differentiation, control temperature, control moisture diffusion, provide chemicals to enhance stem cell growth to the cell line desired, and control the location and direction of growth.

The fiber preferably is in a micron- and submicron-size range and may include a beaded fiber, particle and/or capsule. For example, the fiber may contain an entrapped therapeutic agent. An agent preferably coats the external surface and open pores of the fiber. An agent is an active ingredient deposited on or into the fiber. The fiber may be prepared in a one stage or in multiple stage processes.

The biological materials are preferably an electrohydrodynamic coating of growth factors or other biological materials. Examples are Platelet Growth Factor or Platelet Lysate, EGF, FbF or GMSCF growth factors. The nonbiological material may be drugs or therapeutic agents such as antibiotics. An example of an antibiotic is amoxicillin. There are other well known naturally-derived therapeutic agents obtained from plants.

The fiber may be used alone or it may be added onto a carrier, such as a polymer or other substrate containing additional agents. Preferably polymers are Polyethylene Oxide (PEO) Based Polymer, co-poly lactic acid/glycolic acid (PLGA) and chitosan-based polymer.

Use of a carrier is appropriate when needed to control physical and chemical properties such as temperature, moisture, therapeutic agent release, biochemical agent release, and attract cells such as STEM cells.

Agents coating the fiber or loaded inside or on the carrier can foster cell growth which can be directed and controlled to generate microscopic applications of cell colonization. Cell colonization in turn leads to three-dimensional (3D) structures of tissue and organs. Applications for such 3D structures include research, diagnostics, prognosis applications, and 3D cell simulation systems. Macroscopic applications of such 3D structures maximize skin treatment and/or maximize healing of chronic and acute wounds, burns, and injuries. The fibers and carriers may be combined with a backing material, dressing, and/or bandages to help minimize scaring and the adverse effects of treatment and/or skin rejections.

The coating is preferably an EHD coating and is preferably added in a pattern or morphology that guides cell growth over the surface of the acellular matrix. When a carrier is used, for example, a square geometry of the carrier may be employed where the fiber in or on the carrier is in the order of 2 nm to 100 μm in size.

The coating may also be a random coating alone or on a carrier with fiber preferably of the order of 2 nm to 100 μm in diameter with varied fiber thickness.

If beaded fiber is employed, the beaded fiber diameter preferably ranges from about 25 nm to 100 micron, wherein the dimensions of the beaded fibers determine the release rate.

An embodiment of the present invention employs Intelligent Acellular Xenogeneic Isomorphic Matrix (IAXIM) and stem cells, for example Mesenchymal stem cells (MSCs), with the fiber. The fiber has at least one entrapped agent, such as a therapeutic agent, another biological and abiologial constituent, and an agent coating such as a monoclonal antibody, such as but not limited to anti-CD44, which is selective for MSCs on the external surface and open pores of the fiber.

### Example Using IAXIMs

IAXIMs may be placed in a closed culture system like those provided by a dischargeable plastic flask or box-like cartridge with ports on two opposing sides. One port connects to a sterile plastic tube that connects to metal cannula. The other port connects to another plastic sterile tube that connects to an aspiration pump. Multiple flasks or cartridges may be interconnected in series to enable use of one tube with cannula at one end of the series and a common aspiration pump at the other end. In this way and using a pre-fabricated closed system with IAXIMS, a bone marrow, an umbilical cord, or peripheral blood may be automatically aspirated using the cannula. This multiple-flask setup provides closed systems where the IAXIMS may be selectively attached to improve growth and differentiation of stem cells such as Mesenchymal Stem Cells. Such closed systems with IAXIMS can be cultured in situ for 24 hours and then implanted to the patient in circumstances like those observed in battle fields or sent to the laboratory for further processing.

External heat or energy may be added to the coated acellular matrix to stimulate the release of biological materials from the fibers and encourage cell growth. If a closed system of multiple flasks, as described in the above example, is used, then heat may be applied selectively to one or more flasks.

A method of the invention therefore includes a step of heating the coated acellular matrix to stimulate the release of one or more agents from the fibers and encourage cell growth (160). When heating is involved, the fiber diameters may be varied depending on the subsequent steps anticipated to encourage the release of biological material from the fiber. Heating for example, may take place by subjecting the acellular matrix to radiofrequency (RF) radiation.

Enhancement of RF heating may involve the addition of a magnetic material, such as magnetite, to the fiber coating the acellular matrix, preferably by EHD. This nonbiological material will localize heating from RF radiation affecting the diffusion coefficient of the biologically active materials in the fiber. When a carrier is used, such localized heating can expand the polymer allowing more biomaterials to transport into the cells added to the acellular material, encouraging their propagation.

When no external heat is to be applied, then, the dimensions of the fiber and/or the carrier will be the predominant determinant of the agent release along with the agent diffusion rate, for example, based on the fiber storage temperature, or carrier polymer porosity and thickness.

The invention includes fibers comprising other combinations of a biological and a nonbiological material. For example, combining the magnetite with a bioenzyme, such as papain, can act as a precleaning process in a wound healing process. Fiber and or a carrier having magnetite may also include papain, for example at a concentration of 0.01 to 10 wt %. This bioenzyme may be used to clean up dead cells that may remain in the acellular structure after decellularization.

It has been found that RF radiation in the range from 100 Hz to 3 MHz will heat magnetite and increase the temperature of a polymer carrier and consequently increase moisture diffusion from the carrier to the cells populating the acellular matrix. If a closed system, as described in the above example, is used, then RF radiation may be applied selectively to one or more flasks.

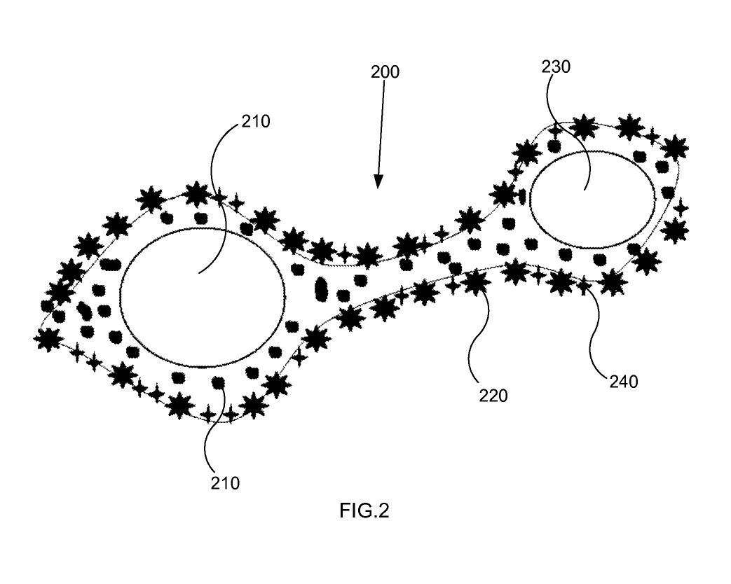

In the step of coating the acellular matrix with a fiber (200), the fiber comprises a biological material and a nonbiological material. Any such material may also be referred to as an agent. An exemplary fiber (200) is shown in cross section in FIG. 2. Solubilized therapeutic molecules (210) are encapsulated within the fiber (200). Cell specific molecules (220) coat the fiber (200). Solubilized growth factor molecules (230) are encapsulated within the fiber (200). Adhesion molecules (240) bind to the exterior of the fiber (200).

An agent used in the fiber comprises one or more of a nutrient, therapeutic, antimicrobial, angiogenesis chemical, antivenom chemical, local anesthetic, adsorbable polyanion, growth factor, buffer salt and polyelectrolyte such as phosphate buffers to stabilize the solution structure of a therapeutic, a selective coating factor (e.g., ligand or antibody), and an extracellular matrix agent. The agents may be on the fiber surface or entrapped and/or dispersed inside particles having at least one biocompatible substance such as a biopolymer as detailed in U.S. Patent Publication US20070296099.

These materials preferably include at least one biochemical that acts as an antimicrobial, such as Acyclovir, Amantadine, Aminoglycosides, Amoxicillin, Amoxicillin/Clavulanate, Amphotericin B, Ampicillin, Ampicillin/sulbactam, Atovaquone, Azithromycin, Cefazolin, Cefepime, Cefotaxime, Cefotetan, Cefpodoxime, Ceftazidime, Ceftizoxime, Ceftriaxone, Cefuroxime, Cephalexin, Chloramphenicol, Clotrimazole, Ciprofloxacin, Clarithromycin, Clindamycin, Dapsone, Dicloxacillin, Doxycycline, Erythromycin lactobionate, Fluconazole, Foscarnet, Ganciclovir, Gatifloxacin, Imipenem/Cilastatin, Isoniazid, Itraconazole, Ketoconazole, Metronidazole, Nafcillin, Nitrofurantoin, Nystatin, Penicillin G, Pentamidine, Piperacillin/Tazobactam, Rifampin, Quinupristin-Dalfopristin, Ticarcillin/clavulanate, Trimethoprim sulfamethoxazole, Valacyclovir, Vancomycin, combinations thereof, and functional equivalents thereof. This biochemical may be of human, animal or recombinant origin, and are preferably in a content range up to about 1 wt %.

The biological and nonbiological material may include at least one biochemical to act as an extracellular matrix agent, such as gycosaminoglycans, hyaluroran, chondroctin sulfate, dermatan sulfate, heparin sulfate, keratin sulfate, collagen, elastra, fibronectin, laminin, combinations thereof, and functional equivalents thereof. This biochemical may be of human, animal or recombinant origin, and may be in a content range up to about 1 wt %.

The biological and nonbiological material may include a local angiogenesis chemical, such as vascular endothelial growth factor (VEGF), acidic fibroblast growth factor (αcFGF), basic fibroblast growth factor (βFGF), heparin sulfate, combinations thereof, and functional equivalents thereof. This angiogenesis chemical may be of human, animal or recombinant origin, and may be in a content range of about 0 to about 1 wt %.

The biological and nonbiological material may include at least one biochemical that acts as a growth factor, such as fibroblast growth factors, G-CSF, GM-CSF, PDGF, TPO, GDF 8, FGF2, βFGF, HGF, combinations thereof, and functional equivalents thereof. This biochemical may be of human, animal or recombinant origin, and may be in a content range of about 0 to about 1 wt %.

The biological and nonbiological material may include at least one biochemical that acts as antimicrobial, such as semisynthetic tetracycline, tetracyclines, rifamycins, polyenes, polypeptides, macrolides, lincomycins, glycopeptides, aminoglycosides, carboxypenems, monobactams, clavulanic acid, beta-lactams (penicillins and cephalosporins), semisynthetic penicillin, combinations thereof, and functional equivalents thereof. This biochemical may be of human, animal or recombinant origin, and may be in a content range of about 0 to about 1 wt %.

The biological and nonbiological material may include at least one biochemical to act as a local anesthetic, such as lipocane, nepivacaine, ropivacaine, artcaine, trimecaine, benzocaine, chloroprocaine, tetracaine, combinations thereof, and functional equivalents thereof. This biochemical may be of human, animal or recombinant origin, and may be in a content range of about 0 to about 1 wt %.

The biological and nonbiological material may be a coating of an adsorbable polyanion. The adsorbable polyanion is preferably any polyanion to which has been conjugated a prosthetic unit intended to increase the hydrophobicity of the polyion. The polyanion may include heparin, hyaluronic acid, chondroitin sulfate, dextran, amino-dextran, dextran sulfate, or modifications of the foregoing such as DEAE-dextran. The prosthetic unit is preferably an alkyl chain, a linear unit containing a benzyl or phenyl group plus at least one dimethylsilane group, or any other prosthetic unit intended to increase the adsorption of the conjugated polyanion to the film. In a preferred embodiment the prosthetic group is composed of linear units containing benzyl-dimethylsilane and may be in a content range of about 0 to about 50 wt %.

The biological and nonbiological material may be a cell growth factor and include a cell-binding moiety that preferentially binds a cell surface molecule, such as a monoclonal antibody, and is preferably selected from the group comprising CD44, CD45, CD90, CD105, CD133, CD34, CD33, CD38, CD105, CD106, nestin, ICAM-I, N-CAM, a selectin, a cadherin, endothelin, V-CAM, P-CAM, collagen, fibronectin, hyaluronic acid, a proteoglycan, TGF-β receptor, and combinations thereof.

The cell growth factor may further include a signaling factor linked to the first cell binding moiety. The signaling factor is preferably selected from the group consisting of EGF, VEGF, NGF, FGF, EPO, G-CSF, GM-CSF, PDGF, IGF-I, stem cell factor, stromal derived factor (SDF-I), MMP inhibitors, LIF, fit-1 ligand, or combinations thereof.

An optional step in an embodiment of the invention is covering the three-dimensional structural shape with a structural layer to enable generation of a replacement external organ; wherein the structural layer comprises: a polymer film; fiber comprising a biological material and a nonbiological material; wherein the biological material comprises a cell growth factor; and, wherein the nonbiological material comprises: magnetite; and, a therapeutic at a concentration between 0.001 and 5 weight percent (150).

The term “therapeutic” refers to a chemical material or compound which, when administered to an organism (human or animal) induces a desired pharmacologic effect. Included are derivatives and analogs of those compounds or classes of compounds specifically mentioned that also induce the desired pharmacologic effect. In particular, the therapeutic may encompass a single biological or abiological chemical compound, or to a combination of biological and abiological compounds that may be required to cause a desirable therapeutic effect. Some examples are compounds used in oncology including those that with epigenetic action or “targeted molecular therapies” such as through inhibition of histone deacetylases (HDACs) and DNA methyltransferases (DNMTs) like valproic acid and or decitabine.

The structural layer controls generation of a replacement external organ, such as skin. A structural layer is preferably used when a limited space will be required to control tissue formation from cell growth and to protect the organ from the environment. The structural layer may also contain an acellular matrix of an organ.

The polymer film is preferably an EHD-coated carrier of the components of the structural layer and is preferably a bioabsorbable transparent polymer. The polymer film preferably comprises a synthetic film such as polyurethane, poly tetrafluoroethylene, extended poly tetrafluoroethylene, copolyester, ethyl vinyl acetate, polyether block amides, polycaprolactone, polylactide, polyglycolide, or cellulose derivative. In a preferred embodiment the polymeric film is ethyl vinyl acetate.

An embodiment of the invention includes a step of covering the coated acellular matrix with fibers on a polymer film wherein the covering is configured to limit growth of a skin organ (170). In this embodiment the polymer film and the fiber covering the acellular matrix are configured to limit growth of a skin organ. Preferably, both the polymer film and the fiber are biodegradable. Embodiments with a structural layer comprising an adsorbable polyanion or a monoclonal antibody like anti CD44, and a bioactive molecule complexed inside the fiber, are useful in the topical treatment of tissue wounds and lesions, especially those of the skin.

In an alternative embodiment, fiber coats polymer film composed of one or more layers made of polyurethane, poly tetrafluoroethylene, extended poly tetrafluoroethylene, copolyesters, ethyl vinyl acetate, polyether block amides, polycaprolactone, polylactide, polyglycolide, cellulose derivatives, combinations of the aforementioned, or the like.

In a preferred method the polymer film is made of ethyl vinyl acetate. The film may be thin and be intended to conform to the application site (conformal) or be adhered to a support structure such as a bandage or compress. The polymer film may be configured or selected to provide features required for treatment. For example, it may be preferably to select a polymer film that is very occlusive and non-breathable, or may be preferable to select a polymer film that is breathable and capable of allowing water transit. As more breathable film is desired, the polymer film will look more like a perforated sheet.

The thickness of the polymer film(s) may be selected for the particular treatment. For example, a thickness in a range of about 0.5 millimeters to about 10 millimeters would be suitable form most treatment applications.

A fiber utilized for a wound dressing might include an absorbent layer in contact with one side of the polymer film. In a preferred embodiment the absorbent layer includes cotton, agar, chitosan, or a combination thereof.

The polymeric film can further be permeable or impermeable to fluids as desired. A fiber coated wound dressing may be made by providing a wound contacting polymeric film with different kind of biodegradable fibers loaded with different molecules like growth factors, cytokynes and or antibiotics. The surface of these nanoparticles and/or nanofibers are preferably coated with different monoclonal antibodies like anti CD44 and special moieties to allowed attaching properties to the film and regenerative stem cells like Mesenchymal stem cells.

If the wound is an internal wound, a wound dressing preferably includes a biodegradable polymeric film. The wound dressing may include a first biodegradable fiber with a bioactive molecule that is an adhesive molecule or a monoclonal antibody with specificity for cells like Mesenchymal stem cells, in this case anti CD44 is preferred, whereby the contacting surface is non-thrombogenic and promotes cellular adhesion.

The bioactive molecule/molecules loaded inside the fiber may include an adhesive molecule, a growth factor molecule, or a therapeutic molecule. The adhesive molecule may be collagen, fibronectin, laminin, vitronectin, thrombospondin, gelatin, polylysine, polyornithine, a peptide polymer, dextran sulfate, a growth hormone, a cytokine, a lectin, or peptidic polymers thereof. The growth factor molecule can be a fibroblast growth factor, platelet-derived growth factor, vascular endothelial growth factor, hepatocyte growth factor, placental growth factor, insulin-like growth factor, nerve growth factor, a neurotrophin, heparin-binding epidermal growth factor, transforming growth factor β, bone morphogenetic protein 2, osteogenic protein 1 or keratinocyte growth factor. The therapeutic molecule can be C—X—C chemokine, interferon gamma, macrophage inflammatory protein-1, an interleukin, IL-1, IL-2, IL-3, IL-4, IL-6, IL-7, IL-8, interferon-gamma inducible protein-10, RANTES, an HIV-tat-transactivating factor, granulocye/macrophage-colony stimulating factor, platelet factor-4 (PF-4), endostatin, angiostatin, amino glycoside antibiotic, streptomycin, gentimicin, tobramycin, neomycin B, actinomycin D, daunorubicin, doxorubicin, bleomycin, rapamycin or paclitaxol. Heparin-activity molecules can also be loaded and include heparin, heparan sulfate, hyaluronic acid, dextran, dextran sulfate, chondroitin sulfate, dermatan sulfate, a molecule including a mixture of variably sulfated polysaccharide chains composed of repeating units of D-glucosamine and either L-iduronic or D-glucronic acids, salts of any of the foregoing, derivatives of any of the foregoing, or combinations of any of the foregoing.

An embodiment of the invention uses fibers containing biological material and nonbiological material comprising fibronectin. The fiber is biodegradable and when assembled according to the method of the invention may be used for contacting surfaces of bandages and other wound-contacting medical films, in order to promote cellular attachment.

A polymer film used in a structural layer may also bind fiber with biological material and a nonbiological material comprising growth factors like FGF-2, VEGF, GM-CSF, bone morphogenic protein-2 (BMP-2) and other molecules like monoclonal antibodies.

A biological material and a nonbiological material may be a bioactive molecule, for example, a molecule intended to increase cell attachment, to modulate biological activity, or to inhibit infections. Examples of molecules intended to increase cell attachment include any of the various types of collagen (type I, type IV, etc.), laminin, fibronectin, fibrinogen, elastin, or amino acid polymers that bind to heparin and support cell attachment.

Examples of molecules that modulate biological activity include heparin-binding growth factors and peptides and analogs derived therefrom, synthetic growth factors, chemokines and modulators of the immune system, and modulators of angiogenesis. Specific examples include any of the known fibroblast growth factors (FGF-1 to FGF-23), the synthetic fibroblast growth factor F2A3, HBBM (heparin-binding brain mitogen), HB-GAF (heparin-binding growth associated factor), HB-EGF (heparin-binding EGF-like factor) HB-GAM (heparin-binding growth associated molecule, also known as pleiotrophin, PTN, HARP), TGF-α (transforming growth factor-α), TGF-βs (transforming growth factor-βs), VEGF (vascular endothelial growth factor), EGF (epidermal growth factor), IGF-1 (insulin-like growth factor-1), IGF-2 (insulin-like growth factor-2), PDGF (platelet derived growth factor), RANTES, SDF-1, secreted frizzled-related protein-1 (SFRP-1), small inducible cytokine A3 (SCYA3), inducible cytokine subfamily A member 20 (SCYA20), inducible cytokine subfamily B member 14 (SCYB14), inducible cytokine subfamily D member 1 (SCYD1), stromal cell-derived factor-1 (SDF-1), thrombospondins 1, 2, 3 and 4 (THBS1-4), platelet factor 4 (PF4), lens epithelium-derived growth factor (LEDGF), midikine (MK), macrophage inflammatory protein (MIP-1), moesin (MSN), hepatocyte growth factor (HGF, also called SF), placental growth factor, IL-1 (interleukin-1), IL-2 (interleukin-2), IL-3 (interleukin-3), IL-6 (interleukin-6), IL-7 (interleukin-7), IL-10 (interleukin-10), IL-12 (interleukin-12), IFN-α (interferon α), IFN-α (interferon-α), TNF-α (tumor necrosis factor-α), SDGF (Schwannoma-derived growth factor), nerve growth factor, neurite growth-promoting factor 2 (NEGF2), neurotrophin, BMP-2 (bone morphogenic protein 2), OP-1 (osteogenic protein 1, also called BMP-7), keratinocyte growth factor (KGF), interferon-γ, inducible protein-20, and HIV-tat-transactivating factor, amphiregulin (AREG), angio-associated migratory cell protein (AAMP), angiostatin, betacellulin (BTC), connective tissue growth factor (CTGF), cysteine-rich angiogenic inducer 61 (CYCR61), endostatin, fractalkine/neuroactin, or glial derived neurotrophic factor (GDNF), GRO2, hepatoma-derived growth factor (HDGF), granulocyte-macrophage colony stimulating factor (GMSCF), C—X—C chemokines, C—X—C chemokines lacking the ELR motif (ELR—C—X—C chemokines), endostatin, angiostatin, and peptide analogs or mimetics thereof, and the many growth factors, cytokines, interleukins and chemokines that have an affinity for heparin. Examples of molecules intended to inhibit infections include amino glycoside antibiotics such as gentimicin, tobramycin and neomycin, and other antibiotics that carry charge groups and in the range of 10 nanograms per milliliter to 100 milligrams per milliliter.

Preferred embodiments using polymer film containing biological material and nonbiological material that is bioactive may be used to treat full-thickness wounds, abrasions, chronic wounds, diabetic ulcers, pressure ulcers, venous stasis (circulatory) ulcers, infections, cuts, incisions, burns, or other surface lesions arising from trauma or disease. In addition to surface lesions, these embodiments may be used internally, such as over soft tissue lesions, organ injuries, around bone, teeth, cartilage or other structures, and as packing material for deep tissue wounds.

A “wound” as used herein includes any of the foregoing. For use internally, it is preferred to use a biodegradable film, such as for example, a polycaprolactone or polylactide. One particularly attractive clinical application is the treatment of lower extremity diabetic neuropathic ulcers that extend into the subcutaneous tissue or beyond and have an adequate blood supply. In the case of treatment of ulcers, the film of this invention can be used as an adjunct to ulcer care, and employed following initial debridement, pressure relief, and infection control. The application of IAXM can be capped or covered in the wound treatment application. Uncovered applications of the IAXM can be used for diagnostics, tumor tissue engineering, or cell culture simulations.

In an alternative embodiment, the polymer film is made of ethyl vinyl acetate and the fiber is made of PLGA with or without iron coating and/or monoclonal antibody anti CD44. The bioactive molecule is recombinant fibroblast growth factor-2 (FGF-2) and/or GM-CSF. In another embodiment the bioactive molecule is the synthetic FGF-2 analog designated F2A3. In the case of F2A3, the film will have biological activity similar to FGF-2, which includes promoting the chemotactic recruitment and proliferation of cells involved in wound repair and enhancing the formation of granulation tissue.

The adsorbable polyanion and the bioactive molecule may be applied to the polymer film serially or in combination, but preferably serially, and can be applied by immersion, spraying, painting, or the like. In one embodiment, the polymer film is immersed in an aqueous solution of 60% isopropanol (v/v) containing 0.25% silyl-heparin (w/v) for 15 minutes at 37 degrees Centigrade. Unbound material is then rinsed with water and the film is air-dried at 56 degrees Centigrade. The polymer film with adsorbed silyl-heparin is then immersed in phosphate buffered saline containing 100 nanograms per milliliter of a bioactive molecule, such as for example F2A3, for 30 minutes at 37 degrees Centigrade and air-dried. The films are then place in foil pouches and sealed. The films may be prepared to be aseptic with low bioburden or be sterile.

Polymer film may be perforated to improve permeability by heating them by RF radiation as some biodegradable fiber distributed inside the film are dissolved faster allowing transit of gases and fluids, and the like. For example, the polymer film is about 0.5 to 10 millimeters thick, and preferably about 1 to 2 millimeters thick. The transit of moisture measured as the moisture vapor transfer rate (MVTR) is from 0-10,000 grams per square centimeter per 24 hours, and preferably is below 2500 grams per square centimeter per 24 hours. The film may be composed of Saran RTM, SARAN HB, SARANEX, ACLAR, polyester, metalized polyester, nylon, metalized nylon, polyvinylidene fluoride copolymer (PVDC), PVDC-Nylon, PVDC-coated polyester, polyvinylchloride, polycarbonate, polystyrene, polyethylene, polyurethane, copolyesters, ethyl vinyl acetate, polyether block amides, poly tetrafluoroethylene, or biodegradable films composed of polycaprolactone, polylactide, or the like.

A preferred method of the invention comprises a step of storing the covered acellular matrix for a time sufficient to form organized cell growth in the three-dimensional structural shape (140). This step may be performed by storing the covered acellular matrix on wounds where the contacting surface attaching forces may be varied as desired.

In embodiments using polymer film, the fiber may contain a thermosensible nonbiological material that contracts as temperature is elevated, and, thus, the particular attachment properties of the polymer film during the storing step can be modified.

### Experiment Using Method

Complex organs like kidney and liver were obtained from an especially biologically controlled and healthy pool of 2-month-old pigs. These xenogeneic organs were made acellular by physical and biochemical methods transforming them into IAXIMs. Their gross and microscopic original architecture was used throughout the process. 3D biomatrices obtained by these means were photographed and studied using light and scanning electron microscopy.

Primary MSCs were obtained from the bone marrow of a healthy 2-month-old rabbit, expanded in vitro using DMEM medium with selected fetal bovine serum, and used at passage 3 cultures for all experiments. MSCs were co-cultured by triplicate within small pieces (1 cubic centimeter) of pig kidney and liver acellular matrices.

MSCs were suspended at a density of 4 million cells per milliliter in a human fibrinogen liquid fraction and infused inside these structures while simultaneously a bovinc thrombin liquid component was injected to polymerize the mixture inside the tissue or the organ matrix. Controls were 1 cubic centimeters pieces of only fibrin with MSCs. A total amount of 2 million cells per construct were used.

These 3D composites were then incubated in a rotating cultivation system. We investigated the effect of the biomatrices on MSC proliferation and differentiation phenotype at days 2 and 21 after initiation of culture. After these times, histological examination was performed in all tissues and controls using light and SEM.

Expression of tissue organ-specific markers for kidney epithelium (pan cytokeratine) and liver epithelium (alpha-fetoprotein, cytokeratines 18 and 19) was determined using immunocytochemistry.

Decellularization led to cell-free scaffolds with preserved extracellular matrix. These matrices supported 3D growth of MSCs in vitro, following embedding in fibrin gel.

At day 2 after initiation of the cultures, none of the cells expressed any of the markers investigated either in the IAXIMs or the controls. The particular 3D topology of each biomatrix facilitated adhesion and proliferation at this time. At day 21 SEM observations confirmed the formation of 3D constructs and the immunohistochemical detection of lineage specific marker molecules showed the presence of cells differentiating into specific cell types inside the IAXIMs but not in the controls.

MSCs seeded in these IAXIMs committed to diverse phenotypes expressing in their surface organ-specific markers of tissue differentiation. At this time, these markers confirmed a kidney or liver character of the composites. Under these conditions the majority of the cells in the acellular liver matrices constructs showed a homogeneous expression of hepatocyte-like phenotype, as revealed by positive immunostaining for alpha-fetoprotein, cytokeratine 19, and cytokeratine 18.

Inside the kidney matrices a very intense positive staining of cell differentiation into tubular-like epithelial cells (expressing the epithelial marker pan cytokeratine) was found.

Matrices from natural sources can be obtained in unlimited amounts, especially from pigs. It was concluded that there are true “matrix superhighway configurations” in their microenvironment with particular biochemical, electrical, and molecular properties that are tissue and organ specific influencing cell differentiation and organogenesis are facilitated with fiber. They should be fundamental for the in vitro regeneration of complex organs for transplantation.

Because these matrices are made acellular they are non-immunogenic, providing excellent isomorphic scaffolds for tissue and organ regeneration. New and interesting specific biochemical and molecular pathways for MSC differentiation are surely inside each one of these matrices and can be enhanced with fiber. Because they are seeded in vitro with MSCs suspended in a fibrin gel that is polymerized in situ, they offer the advantage of a specific 3D architecture for cell growth and differentiation while fibrin maintains the cells in place collaborating in its nutrition.

Cells were identified with tissue-specific markers committed to different lineages inside each of these matrices depending on their physical microenvironment. The evidence indicates that MSCs isolated from bone marrow can differentiate toward a kidney-like or liver-like phenotype inside specific IAXIMs.

Findings suggest that MSCs cultured in the right matrix could differentiate into a mature renal or liver structure with the potential to replace their lost function. Furthermore, in composite 3D cultures like these ones, the precise isomorphic acellular matrix and its specific configuration could permit the development of any tissue type from one common and probably “universal” nonimmunogenic single cell population of MSCs.

These particular specific repertoires are called “matrix superhighway configurations” because their microenvironment appearance and behavior resemble a real interconnected network of highly specialized avenues with specific programs each, where stem cells can grow, differentiate, and produce new tissues and organs as desired.

These “superhighway configurations” are maintained in each of the organ matrices used in this study after they are made acellular and they probably involve Type I collagen, elastin, and a few more macromolecules as well as their specific spatial conformational associations, pigment content, and electrical charges all embedded in a ground matrix consisting of proteoglycans and water that should have the ability to support and differentiate function and polarity of cultured adult MSCs, committing them to the fate of that particular organ.

The main goal should be to provide a culture configuration or scaffold with fiber that more closely mimics the desired tissue or organ in vivo environment. Culturing MSCs in these specific IAXIMs can allow their differentiation within an “embryonic-like niche,” which could lead to specific organogenesis.

For clinical applications, it was determined that these substitutes should be implanted in vivo, not as whole organs, but preferably as “repairing units” using fibrin glue with MSCs to fix them to the surface of a partially resected resident diseased organ hoping to obtain vascularization from the host circulation. The extracellular matrix definitely conducts the regenerative capacity of MSCs, giving new possibilities for organ and tissue regeneration.

The examples given above are merely illustrative and are not meant to be an exhaustive list of all possible embodiments, applications or modifications of the invention. Thus, various modifications and variations of the described methods and systems of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention which are obvious to those skilled in the polymer sciences, molecular biology or related fields are intended to be within the scope of the appended claims.

### INDUSTRIAL APPLICABILITY

The invention has application to the medical services industry and industries involved in the delivery of care and treatment of disease and injuries, including for example, diagnostic and therapeutic oncology.

What is claimed is:

1. A method for organizing and controlling cell growth on a cell-free structure comprising the steps of:
decellularizing a biological material to produce an acellular matrix; wherein the acellular matrix is in a three-dimensional structural shape desired for cell growth;
adding cells sought to be propagated to the acellular matrix;
coating the acellular matrix with a fiber comprising a biological material and a nonbiological material; and,
storing the covered acellular matrix for a time sufficient to form organized cell growth in the three-dimensional structural shape.

2. The method of claim 1 further comprising the step of covering the three-dimensional structural shape with a structural layer to enable generation of a replacement external organ;
wherein the structural layer comprises:
a polymer film;
fiber comprising a biological material and a nonbiological material;
wherein the biological material comprises a cell growth factor; and,
wherein the nonbiological material comprises:
magnetite; and,
a therapeutic at a concentration between 0.001 and 5 weight percent.

3. The method of claim 2 wherein the structural layer further comprises an acellular matrix of an organ.

4. The method of claim 1 for regenerating skin organ further comprising the step of covering the coated acellular matrix with fibers on a polymer film wherein the covering is configured to limit growth of a skin organ.

5. The method of claim 1 further comprising the step of heating the coated acellular matrix to stimulate the release of an agent from the fibers and encourage cell growth.
